(19) United States
(12) Patent Application Publication
(10) Pub. No.: US 2006/0095107 A1
(43) Pub. Date: May 4, 2006

(54) Flexible lead body for implantable stimulation leads

(76) Inventor: Thomas P. Osypka, Palm Harbor, FL (US)

Correspondence Address: EDWARDS & ANGELL, LLP, P.O. BOX 55874, BOSTON, MA, 02205, US

(21) Appl. No.: 11/261,304

(22) Filed: Oct. 28, 2005

Related U.S. Application Data
(60) Provisional application No. 60/622,864, filed on Oct. 28, 2004.

(51) Int. Cl. A61N 1/05

(52) U.S. Cl. 607/122

(57) ABSTRACT

Disclosed is an implantable stimulation lead. The lead includes an inner lead body portion and an outer lead body portion. An interior passage is defined between the inner and outer lead body portions for accommodating a plurality of conductors. A plurality of circumferentially disposed, elongated concavities are defined by either a radially outer surface of the inner lead body portion or a radially inner surface of the outer lead body portion, each concavity for accommodating at least one of the plurality of conductors.

### CROSS-REFERENCE TO RELATED APPLICATION

The subject application claims the benefit of priority from commonly-owned, co-pending U.S. Provisional Patent Application Ser. No. 60/622,864, filed on Oct. 28, 2004, the disclosure of which is herein incorporated by reference in its entirety.

### BACKGROUND OF THE INVENTION

1. Field of the Invention

The subject invention is directed generally to implantable medical devices, and more particularly, to a flexible lead body for an implantable stimulation lead, such as a cardiac pacing and/or defibrillation lead.

2. Background of the Related Art

Implantable electrical leads are utilized in a variety of therapeutic procedures, including neurostimulation for the treatment of chronic pain, electrical sacral nerve stimulation for bladder control, and cardiac pacing and defibrillation for treating arrhythmia. These leads typically consist of one or more conductors extending through an elongated body, the conductors allowing for electrical contact between a portion of the body and a remote device. The remote device is often a signal generator, delivering electrical energy to the body, as well as a diagnostic system for monitoring bodily functioning.

In the particular case of cardiac treatment, the associated implantable leads are an integral component of a life-sustaining process. It is therefore essential that they operate reliably for extended periods of time. Along these lines, cardiac leads are often designed to have coiled electrical conductors rather than straight, thereby increasing the deformation the lead can accommodate before failure and the reliability lifetime of the leads. An example of leads utilizing coiled conductors is disclosed in U.S. Patent Application Publication No. 2005/0060013 to van den Nieuwenhof et al., the disclosure of which is incorporated herein by reference in its entirety. In many cases, respective conductors connect to anodic and cathodic terminals of a remote electrical device, and it is therefore necessary to isolate these conductors from one another. This may be accomplished by individually insulating the coiled conductors; an example of a lead incorporating individually-sheathed conductors is shown in U.S. Patent Application Publication No. 2003/0092303 to Osypka, the disclosure of which is incorporated herein by reference in its entirety. Alternatively, lead conductors can be isolated by incorporating two coil conductors of differing diameter arranged coaxially within the lead body and using insulation tubing to separate the coils.

While cardiac leads utilizing coiled conductors are well suited to accommodating deformation, the coiled shape of the conductors increases their length, and in turn the total resistance of the lead. This increased resistance makes these leads inappropriate for use in applications requiring low resistance conduction paths, such as defibrillation. Instead, defibrillation leads typically employ as the electrical conductors multi-stranded conductor cables (often formed of MP35N or drawn filled tubing), each cable extending through a respective lumen of an insulating, multi-lumen tube. The stranded cables provide a low resistance conduction path, and the multi-lumen tube allows the different cables to be electrically isolated. However, without using a coiled construction for the conductors, such leads tend to fail prematurely due to breaches of the insulation surrounding the conductors and crushing of the conductors themselves. These failures are believed to be caused by stresses developing around the conductors during bending of the lead.

One response to the described failure mechanisms of defibrillation leads is provided by the SPRINT™ ICD (Defibrillation) Lead, which is manufactured by Medtronic, Inc. of Minneapolis, Minn. This lead includes an insulating, multi-lumen body, a plurality of conductor cables, and a conductor coil. Each conductor is accommodated by a respective lumen. Several extra “compression dispersion” lumens are provided, which remain empty and thereby allow for greater compression of the lead body without failing.

One disadvantage of the structure of the above-described SPRINT™ lead is that the compression dispersion lumens reduce the amount of cross-sectional area available for passing conductors. This limits the number of devices that can be associated with a given lead, possibly necessitating the use of multiple leads to accomplish the same function. Conversely, for a given application, the Medtronic lead must be larger in diameter to incorporate the compression dispersion lumens.

It would therefore be beneficial to have a low-profile lead that allows for significant lateral and flexural deformation and maximizes the usable cross-sectional area of the lead body.

### SUMMARY OF THE INVENTION

The present disclosure is directed to an implantable cardiac pacing and/or defibrillation lead that provides the benefits of significant lateral and flexural deformation capability and increased usable cross-sectional area of the lead. The lead includes an inner lead body portion and an outer lead body portion. An interior passage is defined between the inner and outer lead body portions for accommodating a plurality of conductors. A plurality of circumferentially disposed, elongated concavities are defined by either a radially outer surface of the inner lead body portion or a radially inner surface of the outer lead body portion, each concavity for accommodating at least one of the plurality of conductors.

The present invention provides a lead of increased flexibility, due to the lack of connection between the inner and outer lead body portions and concomitant independent movement of the inner lead body portion and the outer lead body portion. Further, the concavities of the present invention allow for movement of the conductors with respect to the inner and outer lead body portions during transverse and flexural deformations. This reduces the stresses in and around the conductors and increases the mechanical reliability of the lead. Finally, the use of a multi-part lead body and the elimination of the use of individual elongated lumens facilitate manufacturing and assembly of the leads.

It should be appreciated that the present invention can be implemented and utilized in numerous ways, including without limitation as a process, an apparatus, a system, a device, and/or a method for applications now known and later developed. These and other unique features of the system disclosed herein will become more readily apparent from the following description and the accompanying drawings.

### BRIEF DESCRIPTION OF THE DRAWINGS

So that those having ordinary skill in the art to which the present application appertains will more readily understand how to make and use the same, reference may be had to the drawings wherein:

FIG. 1 is a side elevational view of a cardiac pacing and/or defibrillation lead configured for active fixation, the lead having a flexible body constructed in accordance with a preferred embodiment of the present invention;

FIG. 2A is a cross-sectional view taken along line 2A-2A of FIG. 1, showing the substantially coaxial inner and outer lead body portions and the conducting wires extending axially through the inner and outer lead body portions and connecting to separately-formed shocking coils;

FIG. 2B is a cross-sectional view, also taken along line 2A-2A of FIG. 1, of an alternative embodiment of the section illustrated in FIG. 2A, in which the conducting wires continue to form shocking coils;

FIG. 3 is a cross-sectional view taken along line 3-3 of FIG. 2A, illustrating the substantially coaxial inner and outer lead body portions, the outer lead body portion having an inner radial surface that defines a plurality of circumferentially disposed, axially elongated concavities that accommodate conducting wires;

FIG. 4 is a localized perspective view in cross-section taken from FIG. 1 showing the conducting wires extending axially through the circumferentially disposed, elongated concavities and the conductor coil extending axially through the inner lead body portion;

FIG. 5 is a perspective view in cross-section, as presented in FIG. 4, of a lead constructed in accordance with another embodiment of the present invention, showing the conducting wires extending axially through the circumferentially disposed, elongated concavities formed in the outer radial surface of the inner lead body portion;

FIG. 6 is a cross-sectional view taken along line 6-6 of FIG. 5, showing the circumferentially disposed concavities formed by the outer radial surface of the inner lead body;

FIG. 7 is a cross-sectional view of a lead constructed in accordance with an alternative embodiment of the present invention, showing concavities formed such that the radial dimension of the concavities is similar to that of the wires being accommodated by the concavities;

FIG. 8 is a cross-sectional view of a lead constructed in accordance with another alternative embodiment of the present invention, showing concavities formed such that the radial dimension of the concavities is larger than that of the wires being accommodated by the concavities, the concavities completely enclosing the conducting wires contained therein; and

FIG. 9 is a cross-sectional view of a lead constructed in accordance with another alternative embodiment of the present invention, showing concavities formed partially by the outer radial surface of the inner lead body portion and partially by the inner radial surface of the outer lead body portion.

These and other features of the implantable stimulation lead of the subject invention will become more readily apparent to those having ordinary skill in the art from the following description of exemplary embodiments.

### DETAILED DESCRIPTION OF PREFERRED EMBODIMENTS

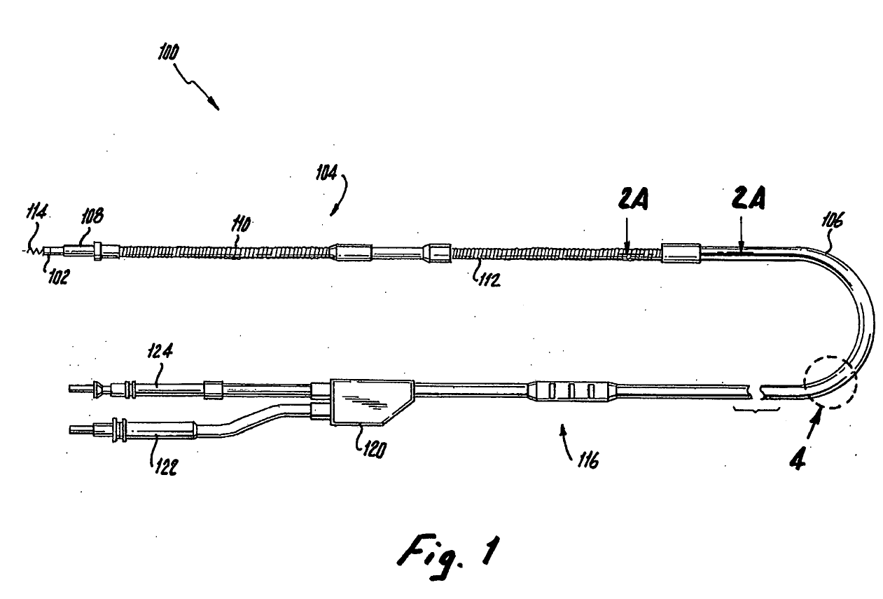

Referring now to the drawings, wherein like reference numerals identify similar structural aspects or features of the subject invention, there is illustrated in FIG. 1 an implantable cardiac pacing and/or defibrillation lead constructed in accordance with a preferred embodiment of the subject invention and designated generally by reference numeral 100. Lead 100 is designed for pacing/sensing and for defibrillation. Accordingly, lead 100 includes a cathodic pacing/sensing electrode 102 operatively associated with the distal end portion 104 of elongated lead body 106, and an anodic pacing electrode 108, a distal shocking coil 110, and a proximal shocking coil 112 all operatively associated with the distal end portion 104 of lead body 106 at a position proximal to the distal pacing/sensing electrode 102. Although anodic electrode 108 and shocking coils 110, 112 are shown arranged in a specific order, these components can be arranged in any order. Preferably, the lead body 106 is composed of a flexible and biocompatible insulating material, such as silicone or polyurethane. Lead 100 is configured for active fixation to the heart, having a retractable/extendible helical fixation screw 114 disposed at the distal end portion 104 for engaging the heart muscle.

A connector assembly 120 is operatively associated with the proximal end portion 116 of the elongated lead body 106 for interacting with an energy-generating device (not shown), such as, for example, an implantable defibrillator or pulse generator/pacemaker. The connector assembly 120 is bifurcated and includes an IS-1 type connector 122, which is electrically connected to the pacing electrodes 102, 108, and a DF-1 type connector 124, which is electrically connected to the shocking coils 110, 112.

Referring to FIGS. 2A, 2B, 3 and 4, the lead 100 includes an outer lead body portion 130 with substantially opposed inner and outer radial surfaces 132, 134. Preferably, outer lead body portion 130 is composed of a flexible and biocompatible insulating material, such as silicone or polyurethane. Inner radial surface 132 defines a lumen 136, and an inner lead body portion 140 extends through lumen 136 such that inner lead body portion 140 is substantially coaxial with outer lead body portion 130. Inner lead body portion 140 has an outer radial surface 144, and the inner and outer lead body portions 140, 130 are configured such that inner radial surface 132 is proximal to outer radial surface 144. Inner radial surface 132 defines a plurality of circumferentially disposed, axially elongated concavities 138. Preferably, inner radial surface 132 is spaced apart from outer radial surface 144 such that there is clearance between the surfaces 132, 144. As shown in the figures, concavities 138 are disposed roughly equidistantly around the circumference of the lead 100. However, in other preferred embodiments, concavities may be distributed in other patterns, or in a non-repeating, arbitrary manner as required.

Paired conducting wires 150a-150d extend axially through lead body 106, the wire pairs 150a-150d being disposed between inner radial surface 132 and outer radial surface 144 and accommodated by the concavities 138. The wire pairs 150a-150d serve to operatively connect the connector assembly 120 and at least some of the electrodes 102, 108 and/or shocking coils 110, 112. For example, one pair of wires 150a provides an electrical connection between the proximal shocking coil 112 and the connector assembly 120. A second pair of wires 150b connects the distal shocking coil 110 and the connector assembly 120. Two more pairs of wires 150c, 150d connect the pacing electrodes 102, 108 to connector assembly 120, as is well known in the art and exemplified in U.S. Patent Application Publication No. 2005/0060013 to van den Nieuwenhof et al. In the embodiment of the invention shown in FIG. 2A, wire pairs 150a, 150b connect to separately-formed shocking coils 110, 112. Alternatively, as shown in FIG. 2B, in another embodiment, wire pairs 150a, 150b themselves form shocking coils 110, 112. The concavities 138 have arcuate and radial dimension sufficient to allow at least one wire to be accommodated therein. The concavities 138 thereby serve to contain wire pairs 150a-150d, such that groups of wires serving distinct electrical purposes are isolated from one another. It should be noted that, while the groups of wires 150a-150d are shown and described as existing in pairs, it is possible to use more or less than two wires in each group.

Inner lead body portion 140 has an inner radial surface 142 that substantially opposes outer radial surfaces 144. Inner radial surface 142 defines a lumen 146. Preferably, inner lead body portion 140 is composed of a flexible and biocompatible insulating material, such as silicone or polyurethane. A pacing conductor coil 152 extends through lumen 146 to provide an electrical connection between at least one of the pacing electrodes 102, 108 and the connector assembly 120. All of the wires 150a-150d are isolated from pacing conductor coil 152 by inner lead body portion 140.

In use, the distal cathodic electrode 102 is used for pacing and as a mapping electrode, sensing electrical potentials. Signals sent by the energy generating device to connector 122 as a function of the sensed electrical potentials propagate to cathodic electrode 102, and continue through the heart, returning through the anodic electrode 108. The shocking coils 110, 112 are designed to deliver electrical energy to cardiac tissue for cardioversion/defibrillation upon demand. While each shocking coil 110, 112 can include a single conductor, it is preferable that at least two conductors be included. This provides lower shocking resistance in the coil and redundancy in case of conductor failure.

By creating concavities 138 in the outer lead body portion 130, the invention obviates the need to individually form a series of specialized conduits to accommodate and isolate the plurality of wires with various electrical functions that are required in a pacing and/or defibrillation lead. This makes the present invention easier and less expensive to manufacture, and presents a significant advantage over the prior art. Another advantage over the prior art is provided by the lack of connection between the inner lead body portion 140 and outer lead body portion 130. This freedom from restraint allows the outer radial surface 144 of the inner lead body portion 140 and the inner radial surface 132 of the outer lead body portion 130 to move independently of one another during transverse loading and bending of the lead, making the lead body more flexible. This lead flexibility is enhanced by the clearance provided between the inner and outer lead body portions 140, 130, which allows the conductors to move with respect to the lead body portions.

Another lead constructed in accordance with a preferred embodiment of the subject invention is illustrated in FIGS. 5 and 6 and designated generally by reference numeral 200. Lead 200 includes an outer lead body portion 230 with substantially opposed inner and outer radial surfaces 232, 234. Inner radial surface 232 defmes a lumen 236, and an inner lead body portion 240 extends through lumen 236 such that inner lead body portion 240 is substantially coaxial with outer lead body portion 230. Inner lead body portion 240 has an outer radial surface 244, and the inner and outer lead body portions 240, 230 are configured such that inner radial surface 232 is proximal to outer radial surface 244. Outer radial surface 244 defines a plurality of circumferentially disposed, axially elongated concavities 238. Concavities 238 are defined by the outer radial surface 244 of the inner lead body portion 240, rather than by inner surface 232 as was described earlier, and accommodate wire pairs 250a-250d. In this embodiment, as in the previous one, concavities 238 extend axially along inner lead body 240, isolating groups of wires with different electrical function from one another. Those skilled in the art will readily appreciate that the number of wires 250a-250d that are situated in each concavity 238 will vary depending upon the application, and are not limited in the present invention.

Inner lead body portion 240 has an inner radial surface 242 that substantially opposes outer radial surfaces 244. Inner radial surface 242 defines a lumen 246, and a pacing conductor coil 252 extends through lumen 246. All of the wire pairs 250a-250d are isolated from pacing conductor coil 252 by inner lead body portion 240.

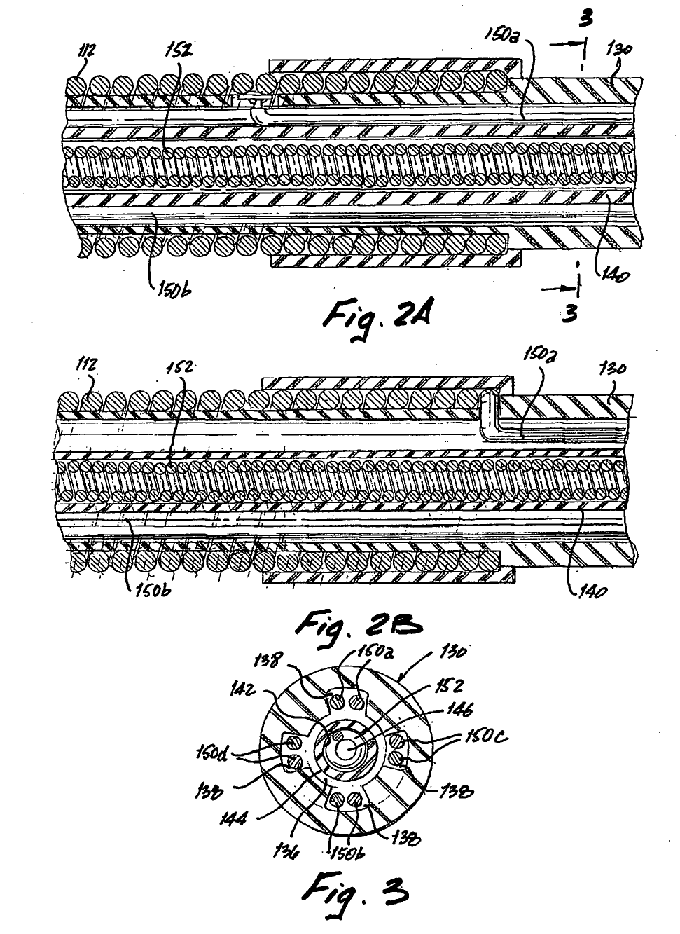

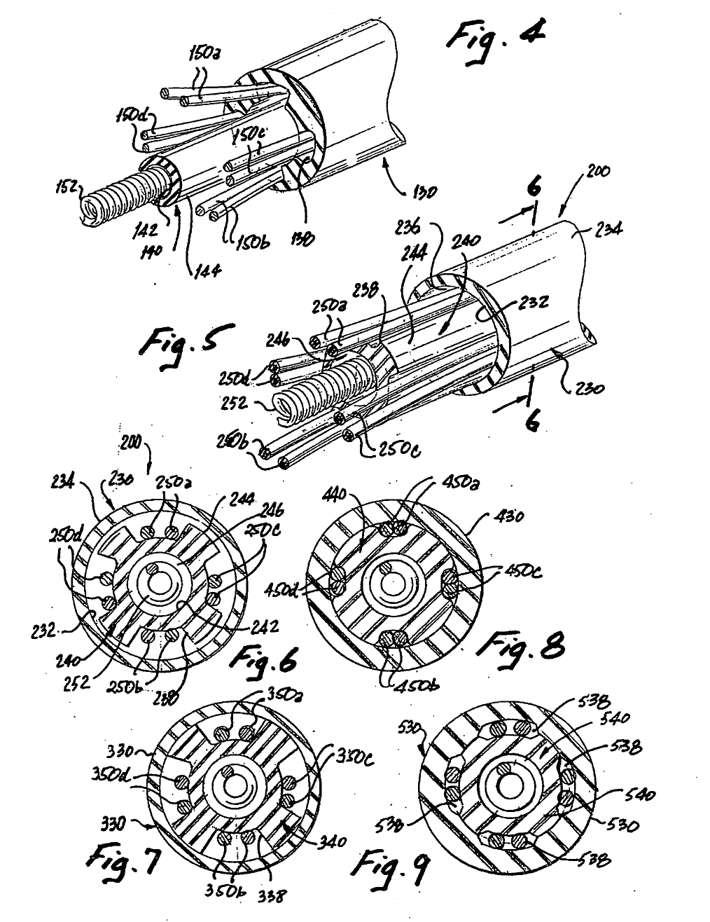

Those skilled in the art will readily appreciate that the previous descriptions are merely exemplary, and that changes and modifications may be made without departing from the spirit and scope of the subject invention. For example, the arcuate and radial dimensions of one or more of the concavities can be varied depending on the application. Referring to FIG. 7, it may be desirable to form concavities 338 in inner lead body portion 340 (or outer lead body portion 330, for that matter) that are significantly larger than the wires 350a-350d contained therein. Alternatively, as shown in FIG. 8, concavities 438 formed in inner lead body portion 440 (or outer lead body portion 430, for that matter) and sized so as to just accommodate wires 450a-450d contained therein may also be utilized. Further, the cross-sectional shapes of any concavities can be varied to accommodate different types of wires or in order to optimize mechanical or some other performance of the lead.

Also, as shown in FIGS. 7 and 8, the inner and outer lead body portions 340, 330, 440, 430 can be dimensioned to eliminate the clearance between these parts, such that different sets of wires 350a-350d, 450a-450d are completely sealed off from one another. Alternatively, the clearance between the inner and outer lead body portions 340, 330, 440, 430 can be increased to increase lead flexibility. Referring to FIG. 9, multiple concavities 538 may be formed partially in the inner lead body portion 540 and partially in the outer lead body portion 530.

In addition, in some applications, it may be desirable to replace conductor cables with coiled conductors. Also, while the axially elongated concavities have been displayed as extending in a direction substantially parallel with the long axis of the lead, the concavities can also be arcuate such that they assume a helical configuration, or can be formed in another non-linear shape. Further, while the lead of the subject invention has been disclosed and illustrated as an active fixation lead having retractable/extendible helical fixation screw, those skilled in the art will readily appreciate that it may alternatively be configured for passive fixation by providing flexible tines at the distal end of the lead body. Finally, although the invention has generally been described in the context of a pacing and/or defibrillation lead, the invention is not limited to use in such applications. Rather, the flexible lead body of the subject invention is more generally applicable to electrical leads for use in any procedure requiring an implantable electrical lead. Such procedures include, but are not limited to, electrostimulation generally (e.g., neuromodulation, neurostimulation, muscle stimulation, and sacral nerve stimulation) and monitoring of bodily functioning.

Thus, while the invention has been described with respect to preferred embodiments, those skilled in the art will readily appreciate that various changes and/or modifications are encompassed by the invention as defined by the appended claims.

What is claimed is:

1. An implantable lead comprising:
a flexible lead body having a longitudinal axis and including an inner lead body portion and an outer lead body portion, the inner and outer lead body portions defining an interior passage therebetween for accommodating a plurality of electrical conductors, wherein at least one of a radially outer surface of the inner lead body portion and a radially inner surface of the outer lead body portion define a plurality of circumferentially disposed elongated concavities, each concavity adapted and configured to accommodate at least one of the plurality of electrical conductors.

2. The lead as recited in claim 1, wherein the outer diameter of the inner lead body is smaller than the inner diameter of the outer lead body, such that the radially outer surface of the inner lead body portion is spaced apart from the radially inner surface of outer lead body portion.

3. The lead as recited in claim 1, wherein the concavities are defined by the inner lead body portion.

4. The lead as recited in claim 1, wherein the concavities are defined by the outer lead body portion.

5. The lead as recited in claim 1, wherein the concavities are defined by both the inner lead body portion and outer lead body portion.

6. The lead as recited in claim 1, wherein each concavity accommodates at least two of the plurality of conductors.

7. The lead as recited in claim 1, wherein the inner lead body portion defines a lumen.

8. The lead as recited in claim 7, further comprising an interior pacing conductor extending through the lumen defined by the inner lead body portion.

9. The lead as recited in claim 8, wherein the lead body is elongated with a proximal and a distal end and the lead further comprises a connector assembly, the connector assembly being secured to the lead body at the proximal end and electrically connected to the pacing conductor and the plurality of conductors.

10. The lead as recited in claim 9, further comprising a pacing electrode, the pacing electrode being secured to the lead body at the distal end and electrically connected to the pacing conductor.

11. The lead as recited in claim 9, further comprising a defibrillation electrode, the defibrillation electrode being secured to the lead body at the distal end and electrically connected to at least one of the plurality of conductors.

12. An implantable lead comprising:
a flexible lead body having a longitudinal axis and including an inner lead body portion and an outer lead body portion, the inner and outer lead body portions defining an interior passage therebetween for accommodating a plurality of electrical conductors, wherein a radially outer surface of the inner lead body portion defines a plurality of circumferentially disposed elongated concavities, each concavity adapted and configured to accommodate at least one of the plurality of electrical conductors.

13. The lead as recited in claim 12, wherein the outer diameter of the inner lead body is smaller than the inner diameter of the outer lead body, such that the radially outer surface of the inner lead body portion is spaced apart from a radially inner surface of outer lead body portion.

14. The lead as recited in claim 12, wherein each concavity accommodates at least two of the plurality of conductors.

15. The lead as recited in claim 12, wherein the inner lead body portion defines a lumen.

16. The lead as recited in claim 15, further comprising an interior pacing conductor extending through the lumen defined by the inner lead body portion.

17. The lead as recited in claim 16, wherein the lead body is elongated with a proximal and a distal end and the lead further comprises a connector assembly, the connector assembly being secured to the lead body at the proximal end and electrically connected to the pacing conductor and the plurality of conductors.

18. The lead as recited in claim 17, further comprising a pacing electrode, the pacing electrode being secured to the lead body at the distal end and electrically connected to the pacing conductor.

19. The lead as recited in claim 17, further comprising a defibrillation electrode, the defibrillation electrode being secured to the lead body at the distal end and electrically connected to at least one of the plurality of conductors.

20. An implantable lead comprising:
a flexible lead body having a longitudinal axis and including an inner lead body portion and an outer lead body portion, the inner and outer lead body portions defining an interior passage therebetween for accommodating a plurality of electrical conductors, wherein a radially inner surface of the outer lead body portion defines a plurality of circumferentially disposed elongated concavities, each concavity adapted and configured to accommodate at least one of the plurality of electrical conductors.

21. The lead as recited in claim 20, wherein the outer diameter of the inner lead body is smaller than the inner diameter of the outer lead body, such that a radially outer surface of the inner lead body portion is spaced apart from the radially inner surface of outer lead body portion.

22. The lead as recited in claim 20, wherein each concavity accommodates at least two of the plurality of conductors.

23. The lead as recited in claim 20, wherein the inner lead body portion defines a lumen.

24. The lead as recited in claim 23, further comprising an interior pacing conductor extending through the lumen defined by the inner lead body portion.

25. The lead as recited in claim 24, wherein the lead body is elongated with a proximal and a distal end and the lead further comprises a connector assembly, the connector assembly being secured to the lead body at the proximal end and electrically connected to the pacing conductor and the plurality of conductors.

26. The lead as recited in claim 25, further comprising a pacing electrode, the pacing electrode being secured to the lead body at the distal end and electrically connected to the pacing conductor.

27. The lead as recited in claim 25, further comprising a defibrillation electrode, the defibrillation electrode being secured to the lead body at the distal end and electrically connected to at least one of the plurality of conductors.
